(19) United States
(12) Patent Application Publication
(10) Pub. No.: US 2022/0039745 A1
(43) Pub. Date: Feb. 10, 2022

(54) IMPACT TRACKING PERSONAL WEARABLE DEVICE

(71) Applicant: IOT MED/DENT SOLUTIONS LLC, Park City, UT (US)

(72) Inventors: Richard Kreifeldt, Park City, UT (US); Robert Miller, Park City, UT (US); Juliana Miller, Park City, UT (US)

(73) Assignee: IOT MED/DENT SOLUTIONS LLC, Park City, UT (US)

(21) Appl. No.: 16/985,524

(22) Filed: Aug. 5, 2020

(51) Int. Cl. A61B 5/00; G08B 7/06; G01L 5/00; G01P 15/18; A63B 71/08; A61B 5/11; H02J 7/02

(52) U.S. Cl. CPC A61B 5/682; G08B 7/06; G01L 5/0052; G01P 15/18; H04B 1/38; A63B 71/085; A61B 5/11; A61B 5/4064; H02J 7/02; A61B 5/0022

(57) ABSTRACT

The present disclosure relates to a personal wearable device. The personal wearable device may include one or more sensors, a charging circuit and a transceiver. In one embodiment, the personal wearable device may be a mouthguard. Also disclosed is a system which includes a personal wearable device and a personal electronic device which receives impact information from the personal wearable device, determines that the impact information indicates that an impact force above a pre-determined threshold has been experienced by the personal wearable device, and transmits a notification.

### BACKGROUND

Sporting events have existed since antiquity although the games or sports being played has changed over the years. Some historians have posited that in many cases sports have been a substitute for training for fighting battles in a war or, alternatively, have helped different regions avoid outright war in favor of playing a game with a clear winner and loser. Sports have always been marked with at least some kind of physical exertion. Frequently, this physical exertion and a desire to win leads at least some players to sustain injuries.

Because of these injuries, many sports have developed rules for protective equipment that is designed to minimize injuries for sport participants. For example, American football started with no helmets or pads which developed into some crudely fashioned padding and leather helmets, which developed into shoulder pads, hip pads, knee pads, and hard plastic padded helmets. Greater emphasis, recently, has been put on the prevention of concussions that are a result of impacts of various pads, body parts, other helmets, and etc. to a person's helmet. Significant rule changes have been implemented in American football from pee-wee leagues to professional football to protect players from concussions. Further, significant research and development has been performed to create more protective helmet padding with the explicit intent of preventing concussions.

American football, which is massively popular in the United States, is a sport in which protective equipment is regularly used. There are, however, other sports where some or no protective equipment is regularly used. For example, basketball, soccer, baseball, rugby, and others utilize minimal protective equipment. As a result, concussions regularly occur in many of these sports due to the impact of a player's head with a foot, elbow, head, or even the ground. Participants in sports with less protective equipment are just as likely to suffer concussions, or even more likely to suffer concussions, than participants in more sports with more direct and intentional physical contact, such as American football.

Concussions have been recognized lately as causing significant damage to a person's brain which may, but does not always, manifest itself in the later years of a sport participant's life. Repeated concussions, specifically, have been associated with chronic traumatic encephalopathy (CTE), which is a neurodegenerative disease. While research is not complete, autopsies of many famous former American football players, in particular, show at least some degree of CTE likely caused by repeated impacts to the players' heads and concussions that they have suffered over the course of their playing years. Many of these players, still alive, have noticed significant cognitive decline in their memory and their ability to communicate in their later years.

Therefore, emphasis has been placed on monitoring players of any sport for any type of concussion with the intent of preventing repeated head trauma to the players. Concussion protocols require, in many cases, a user to discontinue playing the sport after a head impact and physical indications of head trauma, such as an inability to communicate, remember, eye dilation, and other physical manifestations of concussions. Once one or more indications of a concussion have been recognized by team medical staff, the player may not re-enter the sporting event and must clear concussion protocols to participate in subsequent sporting events. Many concussion protocols require an MRI scan or a CT scan to determine whether any physical damage to a person's brain has occurred.

Unfortunately, the ability to monitor a person for a concussion is limited to a medical team's sideline evaluation of the sports participant. Many of these medical teams may be pressured by coaches or other players to allow a player back into the game and clear the player of a concussion. Other times the medical team may not correctly assess subtle indicators that a minor concussion has been experienced by a sport participant. Currently, the only available assessment to identify a concussion is a medical assessment by a medical team on the sidelines.

The state of the art includes some ability to monitor impact tracking. The efforts in this regard have been incorporated into, for example, crash test dummies to identify whether or not a person would be likely to survive a crash in a vehicle. Many of these sensors, which are installed in, for example, a simulated brain cavity, indicate the number of G-forces (measurements of acceleration relative to gravitational forces) experienced by a crash test dummy during a crash test. For example, one or a plurality of sensors may be positioned on or in the head of a crash test dummy. The plurality of sensors may “trip” or indicate that a certain G-force has been experienced by the crash test dummy. The use of a plurality of sensors indicates a maximum level of G-force experienced by the crash test dummy during a crash because if a 4.5G force was experienced, all of the sensors that trip at or below 4.5Gs of force would be tripped while sensors that trip at 4.51 or above will not trip. This provides an indication of an exact, or close approximation of G-force experienced by a crash test dummy during a crash.

Other force sensors are known for use on vehicles themselves, including cars, aircraft, and space vehicles to ensure safe operation for the limitation of human bodies or the expense of hazardous driving, in some circumstances. However, no known solution has been provided for assessing an impact to a live person engaged in the playing of a sporting event.

It is therefore one aspect of this disclosure to provide a device which is wearable that includes one or more sensors for determining an extent of an impact force on a wearer's head or body. It is another object of this disclosure to provide a system that reports the degree of impact force to medical staff, coaches, and parents/guardians/spouses of players to ensure that the best interests of the player are considered and addressed.

### SUMMARY OF THE DISCLOSURE

Disclosed below is a personal wearable device. The personal wearable device may include one or more sensors, a charging circuit and a transceiver. In one embodiment, the personal wearable device may be a mouthguard.

The present disclosure also relates to a system. The system includes a personal wearable device and a personal electronic device which receives impact information from the personal wearable device, determines that the impact information indicates that an impact force above a pre-determined threshold has been experienced by the personal wearable device, and transmits a notification.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1 illustrates a system including a personal wearable device for detecting impacts.

FIG. 2 illustrates a method of collecting impact data and identifying whether a wearer has suffered a concussion.

FIG. 3. illustrates a system that facilitates communication between the personal wearable device and a secondary device.

### DETAILED DESCRIPTION

In the following description of the disclosure, reference is made to the accompanying drawings, which form a part hereof, and in which is shown by way of illustration specific implementations in which the disclosure is may be practiced. It is understood that other implementations may be utilized and structural changes may be made without departing from the scope of the disclosure.

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

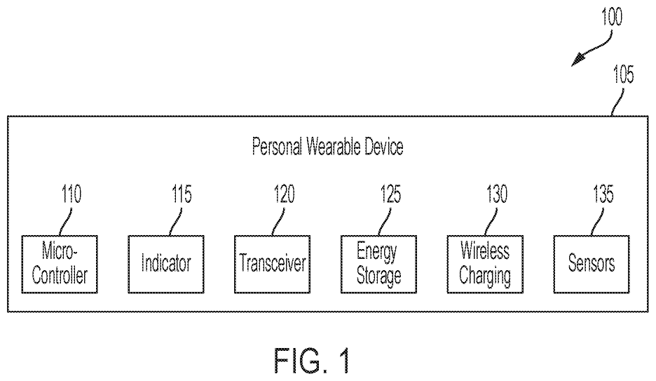

FIG. 1 illustrates a system 100 including a personal wearable device 105. A personal wearable device 105 may include electronic and non-electronic wearable devices. Examples of an electronic personal wearable device may include sporting equipment such as mouth guards, headbands, pads, helmets, and braces for joints fitted with electronic devices described herein. Since a mouth guard is placed within the user's mouth during play, a mouth guard is a preferable implementation to most accurately identify forces to which a brain may be subjected within a player's skull during play of a game. Accordingly, personal wearable device 105 may be implemented as a custom fitted mouth guard with electrical components, described below, which may be disposed within the plastic of the custom fitted mouth guard and which may monitor impact forces and cumulative impact forces on a player's head, neck, and brain during play of a sporting event.

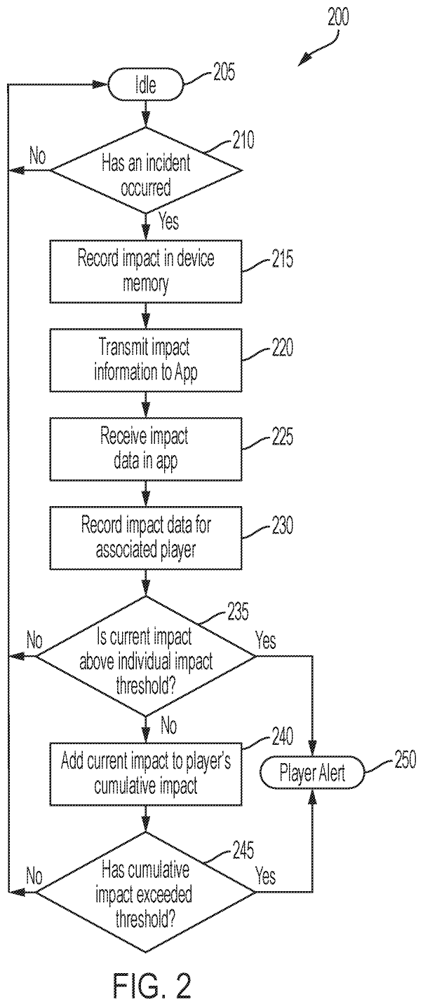

Personal wearable device 105 may include a microcontroller 110, an indicator 115, a transceiver 120, an energy storage 125, wireless charging circuitry 130, and one or more sensors 135 which may all be disposed within personal wearable device 105. Microcontroller 110 may include one or more hardware devices which may include hardware components such as a combination of processors, microcontrollers, busses, volatile and non-volatile memory devices, non-transitory computer readable memory devices and media, data processors, control devices, input devices, output devices, network interface devices, and other types of components that are apparent to those skilled in the art. Microcontroller 110 may execute pre-programmed instructions to perform functionalities described below. For example, microcontroller 110 may execute instructions, stored in a memory device, which cause the microcontroller to accept impact data from the one or more sensors 135, process the impact data, and determine an impact force encountered by personal wearable device 105, as will be discussed below with respect to FIG. 2.

Indicator 115 may be implemented simply as a light (e.g., a light emitting diode) or other display, or may be implemented as an audible indicator such as a piezoelectric speaker. Indicator 115 may operate by interfacing with a personal electronic device, such as a smartphone, tablet, laptop, or desktop computer, and may automatically connect with the personal electronic device to automatically communicate with the device and turn on the indicator which may allow the user to locate personal wearable device 105. Indicator 115 may identify for the wearer of personal wearable device 105 that the player has suffered an impact to the head that exceeds a threshold force level or a cumulative threshold force level and needs to be evaluated for symptoms of concussion.

Personal wearable device 105 may further include a transceiver 120 which may operate to receive instructions to and transmit information from personal wearable device 105. Transceiver 120 may include a number of hardware components such as transmitters, receivers, and antennas. Transceiver 120 may operate using any known network interface communication protocol, including NFC (Near Field Communication), RFID (RF ID tag), Wi-Fi, BLE (Bluetooth Low Energy), ZigBee, Z-Wave, RF (Radio Frequency), RF4CE, Ethernet, telephone line, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, or 802.16m using any network type including a wide-area network (“WAN”), a local-area network (“LAN”), a 2G network, a 3G network, a 4G network, a 5G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between personal wearable device 105 and, for example, a smartphone or cloud based service. Transceiver 120 may or may not be implemented within microcontroller 110 and may or may not be implemented on a single silicon chip.

Energy storage 125 may provide electrical power to other components within personal wearable device 105, such as microcontroller 110, indicator 115, and transceiver 120. Energy storage 125 may be implemented as a battery, a super-capacitor, or any other electricity storage method. In one embodiment, a super-capacitor may be implemented within personal wearable device 105 using various layers that make up the body of the specific personal wearable device. For example, a personal wearable device 105 may be a mouth guard, which is worn in the mouth and is used to cushion teeth from impacting each other (top jaw and bottom jaw) during an impact event. Thus, in the case of a mouth guard, a super capacitor may be built into the retainer by using, in order, at least a biocompatible plastic layer, an electrically conductive layer, an insulating plastic layer, an electrically conductive layer, and a biocompatible plastic layer. Such an organization of various layers of personal wearable device 105 may be similarly implemented in headbands, pads, helmets, and braces for joints, or any other wearable personal electronic device.

Wireless charging circuitry 130 may be implemented using a tightly-coupled electromagnetic inductive coil, a radiative electromagnetic resonant charging circuit, or an uncoupled RF charging. Further, wireless charging circuitry 130 may harvest energy using heat from a person's mouth when installed using thermoelectric or thermionic principles, ambient RF energy, or piezoelectric devices. Wireless charging circuitry 130 may be connected to energy storage 125 and may serve to supply energy to energy storage 125 for storage. Wireless charging circuitry 130 may, under other conditions, also be directly connected to microcontroller 110, indicator 115, and transceiver 120 to provide electrical energy directly to microcontroller 110, indicator 115, and transceiver 120 when, for example, energy storage 125 is depleted of electrical potential or has stored its maximum electrical potential. Further, wireless charging circuitry 130 may be charged by a complimentary charging device which may or may not contain a device which cleans personal wearable device 105 by, for example, exposing personal wearable device 105 to ultraviolet light, particularly light in the UV-C portion of the ultraviolet portion of the electromagnetic spectrum. The charging device may further include an ultrasonic cleaner, and reservoirs to hold cleaning agent, water, and used cleaning agent/water. Personal wearable device 105 may be simultaneously or successively charged and cleaned by the charging and cleaning device.

Personal wearable device 105 may further include one or more sensors 135. One or more sensors 135 may incorporate motion and impact sensors. In one embodiment, sensors 135 may be a multi-axis accelerometer. A multi-axis accelerometer may detect changes in acceleration as a function of time. If personal wearable device 135 is implemented as a mouth guard, for example, one or more sensors 135 may detect forces exerted on the accelerometer which will be virtually identical to forces exerted on the brain of the wearer. Information, including impact information in the form of accelerometer data from sensors 135 may be provided by microcontroller 110 to transceiver 120 for transmitting to a cloud computing server or to a personal electronic device, as will be discussed below.

FIG. 2 illustrates a method 200 of collecting impact data and identifying whether a wearer has suffered a concussion. Method 200 is explained with reference to personal wearable device 105, shown in FIG. 1, and to an associated personal electronic device, which will be described below. Method 200 begins at step 205 where personal wearable device 105 is idle, which means that personal wearable device 105 is waiting for data to be generated from, for example, one or more sensors 135. At step 210, microcontroller 110 detects that an incident has occurred (210—“Yes”) or has not occurred (210—“No”). An incident may be an impact associated with a person playing in a sporting event. If no incident has occurred at step 210 (210—“No”), the device returns to an idle state at step 205. However, if an incident has been detected at step 210 (210—“Yes”), method 200 proceeds at step 215 to record, by microcontroller 110, impact information and acceleration data from one or more sensors 135 in device memory. At step 215, microcontroller 110 may process impact information received from one or more sensors 135 which allows microcontroller 110 to provide a determination of a measured, estimated, or exact force which was applied to personal wearable device 105. Impact information recorded by microcontroller 110 in device memory and/or the determination of the measured, estimated, or exact force applied to personal wearable device 105 may be transmitted by transceiver 120 in personal device 105 to a smartphone application at step 220.

At step 225, a smartphone application associated with a personal electronic device, which will be discussed below, may receive the impact information as impact data. The personal electronic device may further record the impact data in memory for a particular player whose personal wearable device transmitted the impact information at step 230. It should be noted that each one of personal wearable devices 105 in this context may include an identifier of the personal wearable device such that the smartphone associates information received from personal wearable device 105 with a player in a sporting event that experienced the detected impact.

At step 235, the personal electronic device may determine whether or not the impact data indicates an impact over a certain threshold has occurred. In one example, a threshold may be expressed as a certain G-force, a certain acceleration rate, or other units. If the impact data indicates that the experienced impact does not exceed a threshold level (235—“No” pointing left), method 200 returns to step 205. On the other hand, if impact data indicates that the experienced impact has exceeded a threshold level (235—“Yes”), a player alert is generated at step 250 which is a notification that is provided to one or more personal electronic devices indicating that the player has experienced an impact that is likely to have caused a concussion. In one embodiment, the personal electronic devices may be associated with a coach, team medical staff, a referee, umpire, or other official, a player's parents, a player's spouse, or another person who is identified by the player as having a vested interest in the well-being of the player and the ability to make decisions that can protect the health of the player.

If impact data provided to the personal electronic device at step 235 is not above an impact threshold for an individual impact, (235—“No” pointing down), the personal electronic device may add the impact data to a player's cumulative impact level. For example, repeated impacts under the threshold may be as dangerous to the well-being of a player as a single impact that is determined to exceed the threshold. Accordingly, at step 240, the impact data may be added to determine whether or not the impact data indicates that the user has experienced enough impact that a concussion has likely occurred or likely will occur at step 245. If the personal electronic device determines that the cumulative impact data indicates that the cumulative force of impacts during a game (or even a season) is less than a threshold for cumulative impact force (245—“No”), method 200 returns to step 205. However, if the cumulative impact force on a player has exceeded a cumulative impact force threshold (245—“Yes”), a player alert may be generated at step 250. The player alert may be a notification that is provided to one or more personal electronic devices indicating that the player has experienced a cumulative impact force that is likely to have caused a concussion or likely will result in a concussion. In one embodiment, the personal electronic devices may be associated with a coach, team medical staff, a referee, umpire, or other official, a player's parents, a player's spouse, or another person who is identified by the player as having a vested interest in the well-being of the player and the ability to make decisions that can protect the health of the player.

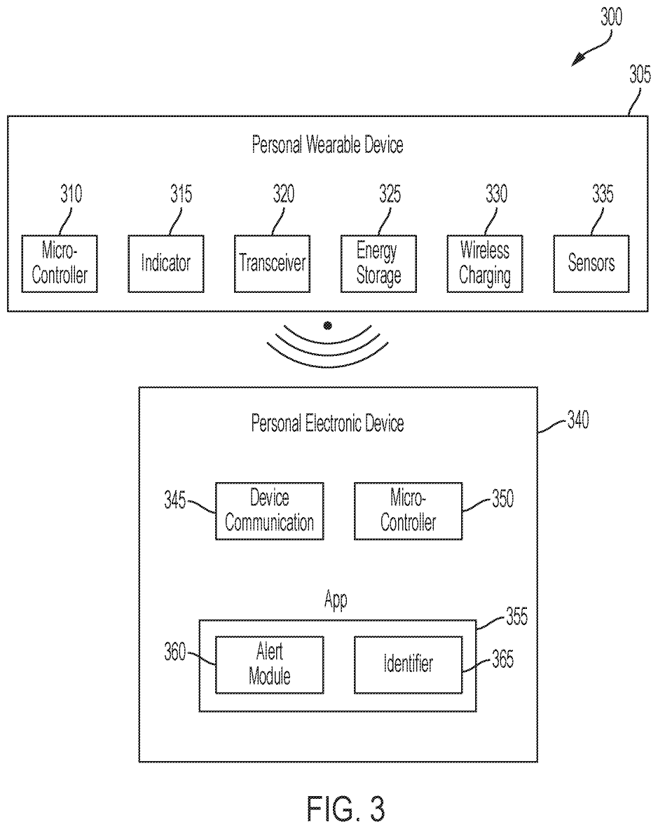

FIG. 3 illustrates a system 300 that facilitates communication between the personal wearable device 305 and a personal electronic device 340. Personal wearable device 305 may be similar in implementation and discussion to personal wearable device 105 shown and described above with respect to FIG. 1. A personal wearable device 305 may include electronic and non-electronic wearable devices. Examples of an electronic personal wearable device may include sporting equipment such as mouth guards, headbands, pads, helmets, and braces for joints fitted with electronic devices described herein. Since a mouth guard is placed within the user's mouth during play, a mouth guard is a preferable implementation to most accurately identify forces to which a brain may be subjected within a player's skull during play of a game. Accordingly, personal wearable device 305 may be implemented as a custom fitted mouth guard with electrical components, described below, which may monitor impact forces and cumulative impact forces on a player's head, neck, and brain during play of a sporting event.

Personal wearable device 305 may include a microcontroller 310, an indicator 315, a transceiver 320, an energy storage 325, wireless charging circuitry 330, and one or more sensors 335. Microcontroller 310 may include one or more hardware devices which may include hardware components such as a combination of processors, microcontrollers, busses, volatile and non-volatile memory devices, non-transitory computer readable memory devices and media, data processors, control devices, input devices, output devices, network interface devices, and other types of components that are apparent to those skilled in the art. Microcontroller 310 may execute pre-programmed instructions to perform functionalities described below.

Indicator 315 may be implemented simply as a light (e.g., a light emitting diode) or other display, or may be implemented as an audible indicator such as a piezoelectric speaker. Indicator 315 may operate by interfacing with a personal electronic device, such as a smartphone, tablet, laptop, or desktop computer, and may automatically connect with the personal electronic device to automatically communicate with the device and turn on the indicator which may allow the user to locate personal wearable device 305. Indicator 315 may identify for the wearer of personal wearable device 305 that the player has suffered an impact to the head that exceeds a threshold force level or a cumulative threshold force level and needs to be evaluated for symptoms of concussion.

Personal wearable device 305 may further include a transceiver 320 which may operate to receive instructions to and transmit information from personal wearable device 305. Transceiver 320 may include a number of hardware components such as transmitters, receivers, and antennas. Transceiver 320 may operate using any known network interface communication protocol, including NFC (Near Field Communication), RFID (RF ID tag), Wi-Fi, BLE (Bluetooth Low Energy), ZigBee, Z-Wave, RF (Radio Frequency), RF4CE, Ethernet, telephone line, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, or 802.16m using any network type including a wide-area network (“WAN”), a local-area network (“LAN”), a 2G network, a 3G network, a 4G network, a 5G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between personal wearable device 305 and, for example, a smartphone or cloud based service. Transceiver 320 may or may not be implemented within microcontroller 310 and may or may not be implemented on a single silicon chip.

Energy storage 325 may provide electrical power to other components within personal wearable device 305, such as microcontroller 310, indicator 315, and transceiver 320. Energy storage 325 may be implemented as a battery, a super-capacitor, or any other electricity storage method. In one embodiment, a super-capacitor may be implemented within personal wearable device 305 using various layers that make up the body of the specific personal wearable device. For example, a personal wearable device 305 may be a mouth guard, which is worn in the mouth and is used to cushion teeth from impacting each other (top jaw and bottom jaw) during an impact event. Thus, in the case of a mouth guard, a super capacitor may be built into the retainer by using, in order, at least a biocompatible plastic layer, an electrically conductive layer, an insulating plastic layer, an electrically conductive layer, and a biocompatible plastic. Such an organization of various layers of personal wearable device 305 may be similarly implemented in pads, headbands, helmets, and braces for joints, or any other wearable personal electronic device.

Wireless charging circuitry 330 may be implemented using a tightly-coupled electromagnetic inductive coil, a radiative electromagnetic resonant charging circuit, or an uncoupled RF charging. Further, wireless charging circuitry 330 may harvest energy using heat from a person's mouth when installed using thermoelectric or thermionic principles, ambient RF energy, or piezoelectric devices. Wireless charging circuitry 330 may be connected to energy storage 325 and may serve to supply energy to energy storage 325 for storage. Wireless charging circuitry 330 may, under other conditions, also be directly connected to microcontroller 330, indicator 335, and transceiver 320 to provide electrical energy directly to microcontroller 330, indicator 335, and transceiver 320 when, for example, energy storage 325 is depleted of electrical potential or has stored its maximum electrical potential. Further, wireless charging circuitry 330 may be charged by a complimentary charging device which may or may not contain a device which cleans personal wearable device 305 by, for example, exposing personal wearable device 305 to ultraviolet light, particularly light in the UV-C portion of the ultraviolet portion of the electromagnetic spectrum. The charging device may further include an ultrasonic cleaner, and reservoirs to hold cleaning agent, water, and used cleaning agent/water. Personal wearable device 305 may be simultaneously or successively charged and cleaned by the charging and cleaning device.

Personal wearable device 305 may further include one or more sensors 335. One or more sensors 335 may incorporate motion and impact sensors. In one embodiment, sensors 335 may be a multi-axis accelerometer. A multi-axis accelerometer may detect changes in acceleration as a function of time. If personal wearable device 335 is implemented as a mouth guard, for example, one or more sensors 335 may detect forces exerted on the accelerometer which will be virtually identical to forces exerted on the brain of the wearer. Information, including impact information in the form of accelerometer data from sensors 335 may be provided by microcontroller 330 to transceiver 320 for transmitting to a cloud computing server or to a personal electronic device 340, as will be discussed below.

Personal electronic device 340 may be implemented as a smartphone, a tablet, a laptop computer, a cloud server computer, or any other device which is capable of wireless communication and executing a program application. Personal electronic device 340 may include device communication circuitry 345 which facilitates information communication between transceiver 320 and personal electronic device 340. Device communication circuitry 345 may execute one or more communication protocols including NFC (Near Field Communication), RFID (RF ID tag), Wi-Fi, BLE (Bluetooth Low Energy), ZigBee, Z-Wave, RF (Radio Frequency), RF4CE, Ethernet, telephone line, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, or 802.16m using any network type including a wide-area network (“WAN”), a local-area network (“LAN”), a 2G network, a 3G network, a 4G network, a 5G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between personal wearable device 305 and, for example, personal electronic device 340, or cloud based service

Personal electronic device 340 may further include a microcontroller 350 which may include one or more hardware devices which may include hardware components such as a combination of processors, microcontrollers, busses, volatile and non-volatile memory devices, non-transitory computer readable memory devices and media, data processors, control devices, input devices, output devices, network interface devices, and other types of components that are apparent to those skilled in the art. Microcontroller 340 may execute pre-programmed instructions to perform functionalities described herein.

Personal electronic device 340 may be programmed with an application 355 which is a series of computer instructions which when executed by microcontroller 350, cause microcontroller 340 to perform a series of actions or a method, such as relevant steps of method 200 discussed above with respect to FIG. 2. Application 355 may include an alert module 360 which may transmit a message using any known communication protocol, including an SMS message, a banner notification, or any other type of notification that an alert has been generated due to an impact of a player identified by an identifier 365 associated with a particular personal wearable device 305. Personal electronic device 340 may be implemented by a plurality of personal electronic devices which are associated with different users concerned about a particular player's well-being. Personal electronic device 340 may trigger an alert through alert module 360 to any person monitoring an identifier 365 associated with a personal wearable device 305 of a particular user. In other words, an alert may be trigged in a personal electronic device 340 associated with one, some, or all of a coach, team medical staff, a referee, umpire, or other official, a player's parents, a player's spouse, or another person who is identified by the player as having a vested interest in the well-being of the player and the ability to make decisions that can protect the health of the player.

In one embodiment, a coach or team medical staff may have access to each player on the team's identifier 365 and may monitor impact information from each player on personal electronic device 340 simultaneously. Impact information received from personal device 305 by any receiver may also be transmitted to the cloud and provided to personal electronic devices 340 directly from the cloud.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A device, comprising:
a mouth guard comprising:
one or more sensors disposed within the mouth guard,
a wireless charging circuit disposed within the mouth guard, and
a transceiver disposed within the mouth guard.

2. The device of claim 1, wherein the one or more sensors include an accelerometer.

3. The device of claim 2, wherein the accelerometer is a multi-axis accelerometer.

4. The device of claim 1, wherein the wireless charging circuit is a radiative electromagnetic resonant charging circuit.

5. The device of claim 1, wherein the wireless charging circuit uses uncoupled radio frequency charging.

6. The device of claim 1, wherein the wireless charging circuit includes an inductive coil.

7. The device of claim 1, further comprising:
an energy storage disposed within the mouth guard.

8. The device of claim 1, further comprising:
an indicator disposed within the mouth guard.

9. The device of claim 8, wherein the indicator indicates an impact force on the mouth guard has exceeded a threshold impact force level.

10. The device of claim 8, wherein the indicator indicates that a cumulative impact force on the mouth guard has exceeded a threshold cumulative impact force level.

11. The device of claim 1, further comprising:
a microcontroller disposed within the mouth guard.

12. The device of claim 1, wherein the one or more sensors provide impact data which is transmitted by a transceiver.

13. The device of claim 12, wherein the impact data is transmitted by the transceiver to a cloud server.

14. The device of claim 13, wherein the impact data is transmitted by the transceiver to a personal electronic device.

15. The device of claim 1, wherein the mouthguard is constructed from plastic.

16. The device of claim 15, wherein the mouthguard is a custom fit mouthguard.

17. The device of claim 1, wherein the transceiver receives information from a personal wearable device and transmits the information to a cloud server through Internet communication circuitry.

18. The device of claim 1, wherein impact information generated by the one or more sensors is stored in electrical components disposed within the mouthguard.

19. A system, comprising:
a personal wearable device, comprising:
one or more sensors,
a wireless charging circuit, and
a transceiver; and
a personal electronic device which receives impact information from the personal wearable device, determines that the impact information indicates that an impact force above a pre-determined threshold has been experienced by the personal wearable device, and transmits a notification.

20. The system of claim 19, wherein the impact information is cumulative impact information, the impact force is a cumulative impact force, and the pre-determined threshold is a cumulative predetermined threshold.
